(19) United States
(12) Patent Application Publication
(10) Pub. No.: US 2013/0333706 A1
(43) Pub. Date: Dec. 19, 2013

(54) Back or Pelvic Belt

(71) Applicant: Hans Bauerfeind, Zeulenroda (DE)

(72) Inventor: Hans Bauerfeind, Zeulenroda (DE)

(73) Assignee: BAUERFEIND AG, Zeulenroda-Triebes (DE)

(21) Appl. No.: 13/985,213

(22) PCT Filed: Feb. 22, 2012
(86) PCT No.: PCT/EP2012/052999

§ 371 Date: Sep. 5, 2013

(30) Foreign Application Priority Data
Feb. 25, 2011 (DE) 102011000953.1

(51) Int. Cl. A61F 5/24

(52) U.S. Cl. CPC A61F 5/24
USPC 128/845

(57) ABSTRACT

The invention relates to a back or pelvic belt having adjacent pads (15, 16, 17, 18) that are attached to a support element (1) in the back area of the belt, with a respective clasp part (4a, 4b) being attached to each of the opposite edge areas (2, 3) of said support element (1) for applying the belt by joining the ends of the two clasp parts characterized in that two tension belts (9a, 9b) are provided on the support element (1) which are attached at the opposite edge areas (2, 3) of the support element (1) and are each guided across the pads (15, 16, 17, 18) in opposite directions such that the pads (15, 16, 17, 18) will approach each other when both tension belts (9a, 9b) are tightened, and will exert a pressure on the affected body part depending on the tensile force acting on the tension belts (9a, 9b).

The invention relates to a back or pelvic belt having two adjacent pads which are attached to a support element in the back area of the belt, a respective clasp part being attached to each of the opposite edge areas of said support element for applying the belt by joining the ends of the two clasp parts.

A belt of this type is disclosed in German published application DE 23 34 500. This prior art belt is used for treating spinal disorders and for alleviating any associated pain. Such pain relief is essentially accomplished through the pads which exert a constant pressure on the muscular tissue on either side of the spinal chord. Such back-to-front pressure provides a constant massage of the back muscles which will thus be strengthened. These pads are held in place by a portion of a belt that can be applied around a person's body and will thus retain their positions at all times.

It is the object of the invention to improve on this prior art belt so as to increase its pain relieving effect. According to the invention, this is accomplished in that the support element has two associated tension belts which are attached to opposite edge areas of the support element and are each guided in opposite directions by means of the pads such that said pads approach each other when both tension belts are tightened, and exert a pressure on the affected part of the body as a function of the tensile force acting on the tension belts.

As a result, this will massage the so-called trigger points in the gluteal muscles, causing the latter to relax. Such muscle relaxation will have a beneficial effect on the positioning of the sacroiliac joint surfaces. The pads approaching each other will have a leverage effect—much like forceps—on the rear protruding part of the ilium, which will in turn have a loosening effect on the sacroiliac joints situated in the inner area of the pelvis. This effect occurs in particular during walking, by the alternating contractions of the gluteal muscles.

The special tension belts and the way they are guided will cause the adjacent pads to be pulled toward each other spatially. As a result, the pads will not only exert pressure toward the inside of the body but will also push on the spinal cord from either side. This will in turn produce a lever arm-like effect which will trigger a repositioning action in the area of the sacroiliac joints if these are in an incorrect position (blockage).

Once the blocked position has been corrected, the tension belts will act to stabilize the sacroiliac joints and the entire pelvic girdle including the symphysis in an advantageous position thereof, thus permanently safeguarding the pain-relieving effect thus obtained.

Tightening of the tension belts can be advantageously accomplished by guiding each tension belt to a tension eyelet and having it terminate in it. A tightening strap each is guided through this tension eyelet and has its one end extending to a clasp part and its other end being free to be grasped so that when the two tightening straps are being tightened, the support element will be contracted, thus causing the pads to approach each other. The free ends of the two tightening straps can be pulled by hand in this way, with the tightening straps exerting a tensile force via the reversing eyelets which is passed on to the tension belts and thus exerts a tensile force in the opposite direction on the support element, causing the support element to be contracted and thus the pads to approach each other.

In order to be able to provide different kinds of pads on the back or pelvic belt, the support element is provided with a hook-and-loop fastener surface which matches the respective hook-and-loop fastener surfaces of the pads, thus allowing different kinds of pads to be exchanged in a simple way via the hook-and-loop fasteners.

Further advantages, features and potential applications of the present invention may be gathered from the description which follows, in conjunction with the embodiments illustrated in the drawings.

Throughout the description, the claims and the drawings, those terms and associated reference signs will be used as are notable from the enclosed list of reference signs. In the drawings

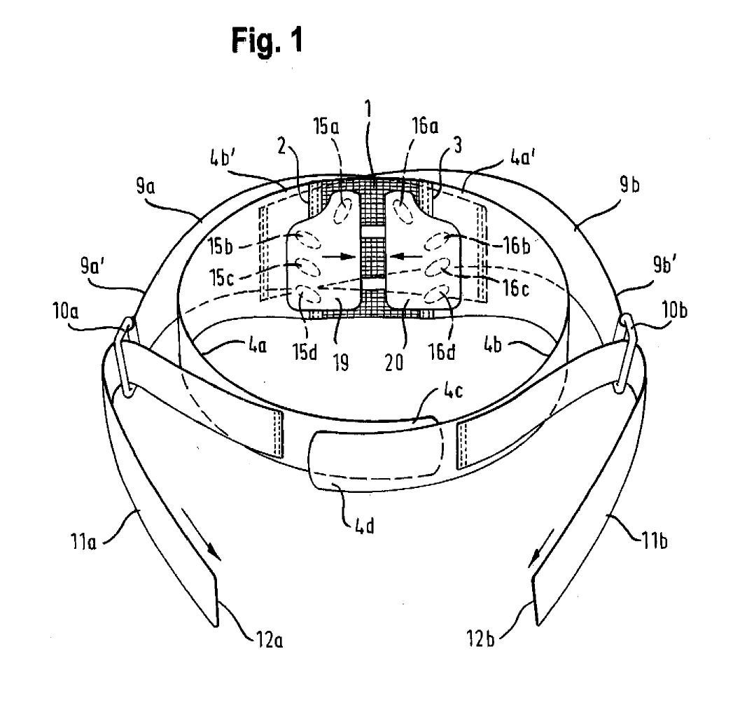

FIG. 1 shows a view of the back or pelvic belt with closed clasp parts and open tightening straps, looking toward the inner surface of the support element;

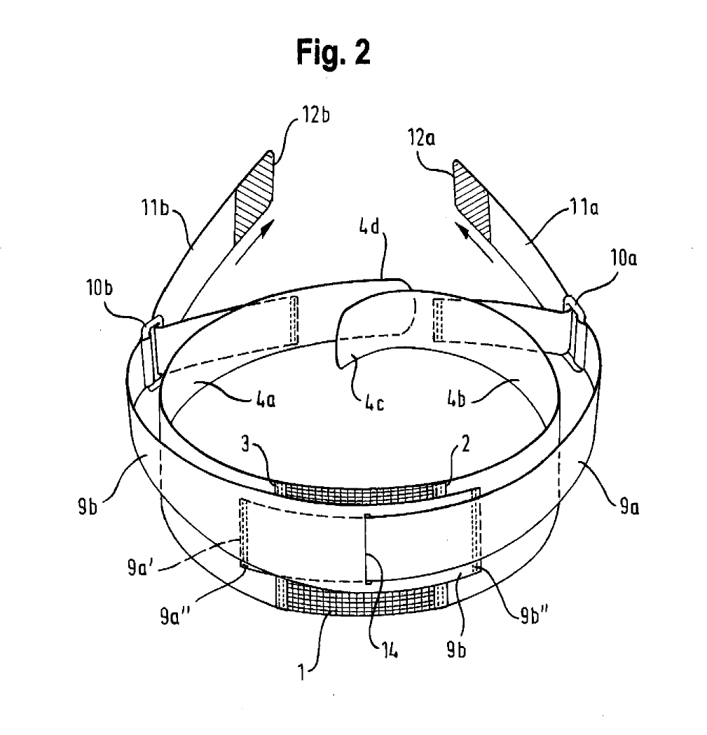

FIG. 2 shows a view of the back or pelvic belt in the same position as in FIG. 1 but looking toward the inner surface of the closed clasp parts;

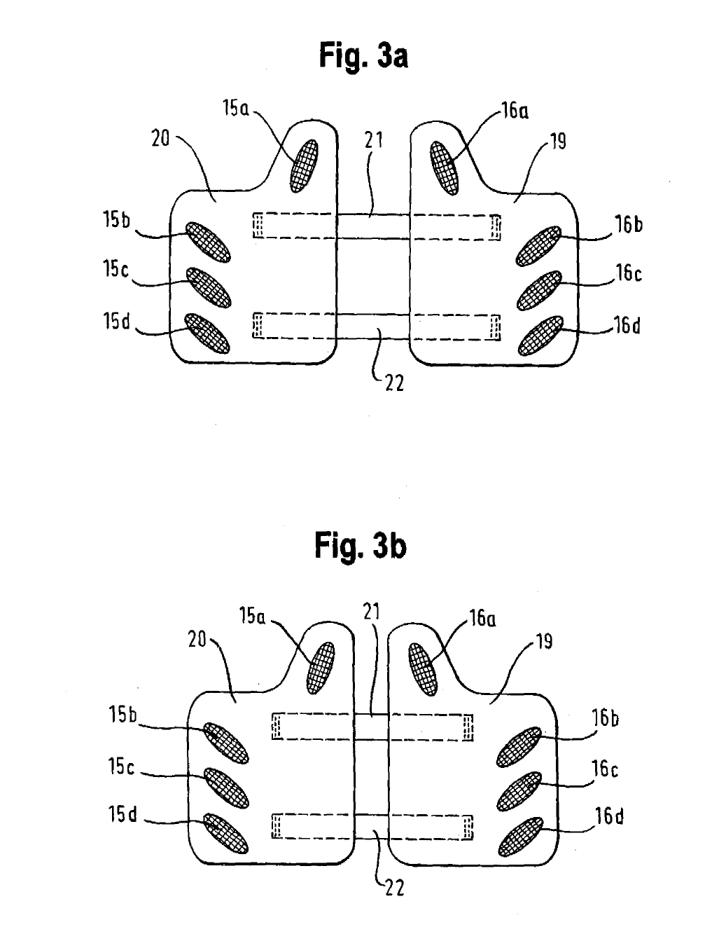

FIGS. 3a and 3b show different views of the support element, with the pads pulled apart and brought closer together, resp.;

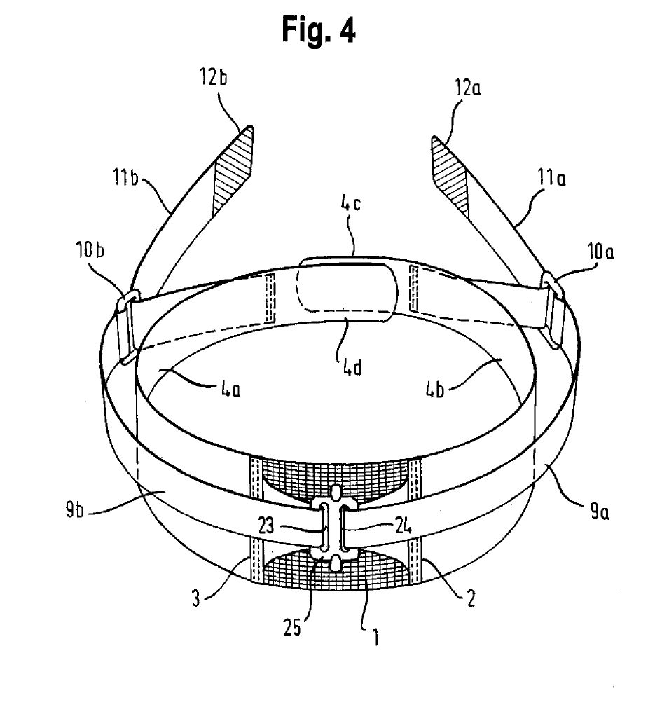

FIG. 4 shows a view of the belt in the same position as in FIG. 2 however including a direction reversal element for guiding the tension belts in opposite directions, and

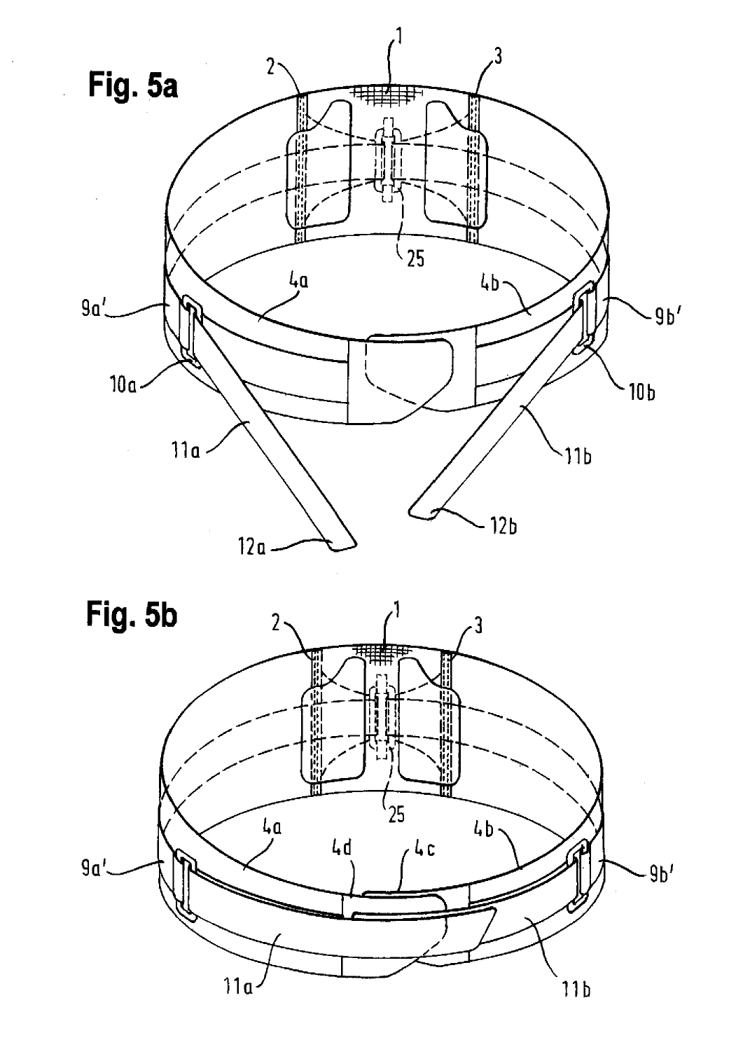

FIGS. 5a and 5b show views of the belt in a position similar to the one of FIG. 1 but with a direction reversal element being provided on the support element, which latter is shown in a released position in FIG. 5a and in a contracted position in FIG. 5b.

FIG. 1 shows the belt with a view of the inner surface of the elastic support element 1 having eight pads 15a, 15b, 15c, 15d and 16a, 16b, 16c, 16d mounted thereon. Attached to either side 2 and 3 of the support element 1 are the ends 4a′ and 4b′ of the two clasp parts 4a and 4b. These are to be passed around a patient's body and then tightened so as to stretch the elastic support element 1. In the clasping position shown, the clasp parts 4a and 4b are made to overlap at their respective ends 4c and 4d and are thus connected to form a closed ring, said ends 4c and 4d being in the form of hook-and-loop fasteners. This makes it possible to open the clasp parts 4a and 4b at any time for taking off the belt and to close them again once the belt has been wrapped around a person's body.

Attached to the side of the support element 1 facing away from the viewer are the ends 9a′ and 9b′ of the two tension belts 9a and 9b. The two tension belts 9a and 9b partially surround the clasp parts 4a and 4b and terminate in the two eyelets 10a and 10b resp., through which a tightening strap 11a and 11b, resp. has been passed. When the tightening straps 11a and 11b are being tightened by pulling on their free ends 12a and 12b in the direction of the arrow, a tensile force will be exerted on the tension belts 9a and 9b much in the manner of a tackle. This tensile force will cause the elastic support element 1 to contract because of the guidance of the two tension belts 9a and 9b in opposite directions as is illustrated in more detail in FIG. 2, or will cause the elastic support element 1 to relax when the tension belts 9a and 9b are released. Once the two clasp parts 4a and 4b are in place, they will be connected with each other via their ends 4c and 4d which are in the form of hook-and-loop fasteners. Consequently, the belt will now be appropriately positioned on a person's body with the support element 1 stretched to a higher or lesser extent and with the pads 14, 15, 17 and 18 in their desired positions firmly pressing against the body. FIG. 2 is a view of the back or pelvic belt of FIG. 1 in a position which shows the tension belts 9a and 9b being guided in opposite directions on the support element 1. Further shown in FIG. 2 is the particular form of attachment of the respective ends 9a′ and 9b′ of the tension belts 9a and 9b on the clasp parts 4a and 4b. The tension belt 9a surrounds the support element 1 and has its end 9a′ firmly connected to the clasp part 4a by means of the seam 9a″. The tension belt 9b initially overlies the end 9a′ of the tension belt 9a which is anchored in the seam 9a″, and contains the slot 14 in the tension belt 9b and will slide next to the slot 14 and finally toward the end 9b′ having the seam 9b″ and will thus be firmly connected to the clasp part 4b. As can be seen, the seam 9b″ is partially covered by the tension belt 9a. When, with this design of the tension belts 9a and 9b terminating in the seams 9a″ and 9b″, the tightening straps 11a and 11b are tightened, this will result in a tensile force guided via the eyelets 10a and 10b and acting in opposite directions on the tension belts 9a and 9b, which will in turn cause the ends 9a′ and 9b′ of the tension belts 9a and 9b which are anchored in the seams 9a″ and 9b″ to be contracted. This tensile force will cause the seams 9a″ and 9b″ on the support element 1 to approach each other. As a result, a force contracting the support element 1 will act on the back or pelvic belt and thus make the back belt tighter.

Shown in FIG. 3a are the pads 15a, 15b, 15c, 15d and 16a, 16b, 16c, 16d on their respective supports 19 and 20 in the same position as shown in FIG. 1, i.e. pulled apart. The two supports 19 and 20 are connected to each other through elastic straps 21 and 22.

FIG. 3b shows the two supports 19 and 20 having the four pads 15a, 15b, 15c, 15d and 16a, 16b, 16c, 16d in a position in which they have been made to approach each other. This position will result from contracting the associated support element 1 (see FIG. 5b) provided that the two tightening straps 11a and 11b have been tightened appropriately.

Shown in FIG. 4 is a back or pelvic belt which is identical to the one shown in FIG. 2 except for the design on the support element 1. As regards this design, reference can therefore be made to the explanations regarding FIG. 2. In the embodiment of FIG. 4, each of the two tension belts 9a and 9b will first be guided through slots 23 and 24, thus having their direction reversed by the reversal piece 25, and then be firmly attached on the respective sides 2 and 3 of the support element 1. When the two tension belts 9a and 9b are tightened, this direction reversal will result in a contraction of the support element 1 and thus cause the pads mounted thereon to approach each other, as is shown in FIG. 3b.

Shown in FIGS. 5a and 5b is the design of the back or pelvic belt of FIG. 4 in a position in which the inner surface of the support element 1 can be seen. To be more precise, the view of FIG. 5a shows the support element 1 in an extended state and the view of FIG. 5b shows the support element 1 in a contracted state which latter state is obtained due to the tightening of the tension belts 9a and 9b. As regards the other components of the belt, reference is made to the illustrations of FIGS. 1 and 2 and their respective explanations. For reasons of clarity, the pads have not been drawn in here as their arrangement can clearly be seen in FIG. 1.

It should be noted that the view of FIG. 5b shows the back or pelvic belt in a closed position thereof, in which the two clasp parts 4a and 4b are illustrated in their closed state. On and above this closed position as shown in FIG. 1 the two tightening straps 11a and 11b are also shown in their closed position. As a result, the entire back or pelvic belt is closed in itself.

### LIST OF REFERENCE SIGNS

1 support element
2 side
3 side
4a clasp part
4b clasp part
4c respective end
4d respective end
4a′ end
4b′ end
9a tension belt
9b tension belt
9a′ end
9b′ end
9a″ seam
9b″ seam
10a tension eyelet
10b tension eyelet
11a tightening strap
11b tightening strap
12a free end
12b free end
14 slot
15 pad
15a pad
15b pad
15c pad
15d pad
16a pad
16b pad
16c pad
16d pad
17 pad
18 pad
19 support
20 support
21 elastic strap
22 elastic strap
23 slot
24 slot
25 reversal piece

1. A back or pelvic belt having adjacent pads (15, 16, 17, 18) that are attached to a support element (1) in the back area of the belt, with a respective clasp part (4a, 4b) being attached to each of the opposite edge areas (2, 3) of said support element (1) for applying the belt by joining the ends of the two clasp parts characterized in that two tension belts (9a, 9b) are provided on the support element (1) which are attached at the opposite edge areas (2, 3) of the support element (1) and are each guided across the pads (15, 16, 17, 18) in opposite directions such that the pads (15, 16, 17, 18) will approach each other when both tension belts (9a, 9b) are tightened, and will exert a pressure on the affected body part depending on the tensile force acting on the tension belts (9a, 9b).

2. The back or pelvic belt of claim 1 characterized in that the tension belts (9a, 9b) are guided to the respective opposite edge area (2, 3) of the support element (1) in an intersecting manner.

3. The back or pelvic belt of claim 1 characterized in that the tension belts (9a, 9b) are each guided through a reversal piece (25) provided between the pads (15, 16, 17, 18) for direction reversal, from where they are then guided to the respective edge area (2, 3) of the support element (1).

4. The back or pelvic belt of claim 1 characterized in that each tension belt (9a, 9b) extends to a tension eyelet (10a, 10b) and terminates therein, through which a tightening strap (11a, 11b) each is guided which has its one end extending to a clasp part (4a, 4b) and its other end left free for grasping in such a way that when the two tightening straps (11a, 11b) are tightened, the support element (1) will contract and the pads (15, 16, 17, 18) will approach each other.

5. The back or pelvic belt of claim 1 characterized in that the support element (1) has a hook-and-loop fastener surface which matches the respective hook-and loop fastener surfaces of the pads (15, 16, 17, 18).
